(19) United States
(12) Patent Application Publication
(10) Pub. No.: US 2026/0015659 A1
(43) Pub. Date: Jan. 15, 2026

(54) Method for Nucleic Acid Sequence Detection and Sequencing Method

(71) Applicants: SEQULITE GENOMICS US, INC., Dublin, CA (US); Guangdong Runpeng Biological Technology Co., LTD., Dongguan City, Guangdong (CN)

(72) Inventor: Xiongwei YAN, Dublin, CA (US)

(21) Appl. No.: 18/770,607

(22) Filed: Jul. 11, 2024

(51) Int. Cl. C12Q 1/6874; C12Q 1/6816

(52) U.S. Cl. CPC C12Q 1/6874; C12Q 1/6816

(57) ABSTRACT

The present disclosure discloses a method for nucleic acid sequence detection and a sequencing method. The method for nucleic acid sequence detection includes: providing a nucleic acid with a preset sequence; and detecting sequence information of the nucleic acid through a hybridization signal between a probe and the nucleic acid.

### SEQUENCE LISTING

The present application contains a Sequence Listing which has been submitted electronically in ASCII format and is hereby incorporated by reference in its entirety. Said ASCII copy is PN223198FPSW_Sequence_Listing.xml and is 12,164 bytes in size. The date of creation is 2024-08-02. Said ASCII copy contains SEQ ID NO: 1 to SEQ ID NO:8.

### TECHNICAL FIELD

The present disclosure relates to the technical field of nucleic acid sequencing, specifically to a method for nucleic acid sequence detection and a sequencing method.

### BACKGROUND

Second-generation sequencing technology, also known as next-generation sequencing technology (NGS), is also called high-throughput sequencing technology due to its high throughput, which can read thousands of short DNA fragments at one time.

An index is a sequence of tags added to distinguish samples from different sources. Thus, the same index tag is added to each sample.

Classical second-generation sequencing mainly includes three steps: library preparation, bridge PCR amplification, and sequencing. The library preparation includes: 1) randomly breaking the DNA to be detected into fragments of a certain length; and 2) processing the two ends of the fragments. As shown in FIG. 1, each resulting DNA single strand has P7, i7′ index, and Rd2SP (Read2 Sequencing Primer) at the 5′ end, and P5′, i5 index, and Rd1SP′ (Read1 Sequencing Primer) at the 3′ end. These DNA single strands form a sample library. Numerous oligonucleotide strands (P5, P7′) are immobilized on the surface of the microarray chip used for sequencing, which can complement and bind to P5′ and P7, respectively.

At present, the i7 index and i5 index are generally sequenced and identified by SBS (sequence by synthesis). However, this kind of sequencing for known sequences still has the problem of high cost and long time.

### SUMMARY

The present disclosure aims to provide a method for nucleic acid sequence detection and a sequencing method, so as to solve the problem of high cost and long time for sequencing known sequences in the prior art.

In order to achieve the above purpose, according to one aspect of the present disclosure, a method for nucleic acid sequence detection is provided. The method includes: providing a nucleic acid with a preset sequence; and detecting sequence information of the nucleic acid through a hybridization signal between a probe and the nucleic acid.

Furthermore, the nucleic acid with the preset sequence has M type(s), with the M≥1, and the probe that is reversely complementary to the M type(s) of the nucleic acid with the preset sequence each carries a preset fluorescent label.

Furthermore, the method includes: S1, immobilizing the nucleic acid with the preset sequence on a solid support; S2, dividing the probe into N group(s), with the N≥1, where each group of the probe includes a type(s) of the probe, with the a ≥1, and the a type(s) of the probe in the group each carries a different fluorescent label; and hybridizing a first group of the probe carrying a fluorescent label with the nucleic acid with the preset sequence to collect a fluorescent signal; S3, stripping the first group of the probe, and hybridizing a second group of the probe with the nucleic acid with the preset sequence to collect a fluorescence signal; S4, repeating the step S3 until all the nucleic acid with the preset sequence is detected; and S5, identifying the sequence information of the nucleic acid with the preset sequence according to presence or absence of the hybridization signal between the probe and the nucleic acid.

Furthermore, the nucleic acid with the preset sequence is an index; and preferably, the length of the nucleic acid with the preset sequence is 8-12 bp.

Furthermore, the fluorescent label includes one or more of AF532, Rox, Cy5, or AF700.

According to another aspect of the present disclosure, a sequencing method is provided. The method includes sequencing of a sequence to be detected with an unknown sequence and sequencing of a preset sequence as an index, where the sequencing of the preset sequence includes identifying sequence information of the preset sequence using any one of the above methods.

Furthermore, the sequencing method is double-ended sequencing or single-ended sequencing; and/or a template strand for sequencing includes one or more preset sequences; and/or the sequencing method uses bridge amplification or rolling circle amplification for sequencing.

Furthermore, a single-stranded template nucleotide for sequencing sequentially includes a first linker sequence region linked to a solid support, a first index region, a first sequencing primer region, a sequence region to be detected, a second sequencing primer region, a second index region, and a second linker sequence region linked to the solid support from a 5′ end to a 3′ end; and the method includes: immobilizing the single-stranded template nucleotide on the solid support through a bridge reaction to form a cluster, removing one of nucleotide strands identical or complementary to the single-stranded template nucleotide by cleaving, and retaining a first sequencing strand; hybridizing a probe carrying a fluorescent label for identifying the first index region with a nucleic acid of the first index region to collect a fluorescent signal and identify a sequence of the first index region, and then stripping the probe; hybridizing a first nucleic acid fragment covering a portion of the first linker sequence region, the first index region and the first sequencing primer region with the cluster, linking the first nucleic acid fragment to a linker sequence on the solid support, and partially sequencing the sequence to be detected with the first nucleic acid fragment as a primer, and then extending the first nucleic acid fragment to the second linker sequence region; removing the first sequencing strand; hybridizing a probe carrying a fluorescent label for identifying the second index region with a nucleic acid of the second index region to collect a fluorescent signal and identify a sequence of the second index region, and then stripping the probe; and performing complementary strand sequencing of the sequence to be detected using a second nucleic acid fragment complementary to the second sequencing primer region as a prime.

Furthermore, removing one of the nucleotide strands identical or complementary to the single-stranded template nucleotide by cleaving includes: cleaving a uracil base or an 8-oxoguanine base on a nucleic acid linker on the solid support to form a nick, and removing a nucleotide strand with the nick using a formamide solution.

Furthermore, the uracil base is cleaved using a uracil hydrolase, or the 8-oxoguanine base is cleaved using an Fpg glycosidase.

Furthermore, the stripping of the probe includes stripping the probe using a formamide solution.

Furthermore, the method further includes determining whether the probe is completely stripped by fluorescence observation.

Furthermore, both a 5′ end and a 3′ end of the first nucleic acid fragment are phosphorylated.

Furthermore, the step of linking the first nucleic acid fragment to a linker sequence on the solid support, and sequencing the sequence to be detected with the first nucleic acid fragment as a primer specifically includes: linking the first nucleic acid fragment to the linker sequence on the solid support using a DNA ligase, dephosphorylating the 3′ end of the first nucleic acid fragment using a phosphatase, and sequencing the sequence to be detected with the first nucleic acid fragment as a primer.

Furthermore, after removing the first sequencing strand, the method further includes a step of blocking a free 3′-hydroxyl group with a terminator or phosphorylation, and optionally, the terminator is a dideoxynucleotide.

By applying the technical solutions of the present disclosure, the sequence of the index is preset, and the index is directly hybridized and distinguished by the probe carrying the label, that is, the sequence information of the index is directly identified by the label signal after the index is hybridized with the probe. In this way, the sequence information of the index can be identified only by the presence or absence of the fluorescence signal, which can greatly shorten the sequencing time and reduce the sequencing cost. Usually, the SBS method takes tens of minutes or even 1-2 hours for index sequencing, while the color development of the fluorescently labeled probe can be completed within a few minutes, so the entire sequencing workflow (i.e., turnover time) can be significantly shortened. Meanwhile, the manufacturing process of fluorescently labeled oligonucleotides is mature, the method is simple, and the price is low. In other words, using the technical solutions of this disclosure, there is no longer a need to sequence the index, and the sequence information of the index can be identified only through inexpensive fluorescence reactions.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings of the specification, which form part of this disclosure, are used to provide a further understanding of the present disclosure, and the illustrative embodiments and their explanations of the present disclosure are used to explain the present disclosure and do not constitute an improper limitation of the present disclosure. In the drawings:

FIG. 1 shows a schematic diagram of a sequencing single-strand structure according to an embodiment of this disclosure;

FIG. 2 shows a schematic diagram of index and probe settings according to an embodiment of this disclosure;

FIG. 3 shows a fluorescence detection image after a first hybridization according to Embodiment 1 of this disclosure;

FIG. 4 shows a fluorescence detection image after a second hybridization according to Embodiment 1 of this disclosure;

FIG. 5 shows a composite fluorescence detection image according to Embodiment 1 of this disclosure; and

FIGS. 6-1, 6-2, and 6-3 show a schematic diagram of a sequencing process according to Embodiment 2 of this disclosure.

### DETAILED DESCRIPTION OF THE EMBODIMENTS

It should be noted that, in the absence of a conflict, embodiments of this disclosure can be hybridized with features in the embodiments. The present disclosure will be described in detail below with reference to the accompanying drawings and in conjunction with embodiments.

The index in sequencing is a tag sequence used to identify nucleic acid samples. During the sequencing process, nucleic acid samples are usually divided into small fragments, and each fragment needs to be labeled for subsequent data analysis.

Original template strand: in this disclosure, the original template strand refers to a nucleotide strand from a sequencing library in sequencing.
sequencing strand: in this disclosure, the sequencing strand refers to a nucleotide strand synthesized during the sequencing process.

As mentioned in the background art, in the existing high-throughput sequencing, the index is generally sequenced and identified by SBS (sequence by synthesis). In order to improve the efficiency of sequencing for this known sequence and reduce the cost of sequencing, the following technical solutions are proposed in this disclosure.

In the technical solution of this disclosure, the sequence of the index is preset, and the index can be directly hybridized and distinguished by the probe carrying the label, that is, the sequence information of the index can be directly identified by the presence or absence of the label signal after the index is hybridized with the probe. Based on this disclosure concept, the specific technical solutions are as follows:

According to a typical implementation of the present disclosure, a method for nucleic acid sequence detection is provided. The method includes: providing a nucleic acid with a preset sequence; and detecting sequence information of the nucleic acid through a hybridization signal between a probe and the nucleic acid.

By applying the technical solutions of the present disclosure, the nucleic acid with the preset sequence is directly hybridized and distinguished by a probe carrying a label, that is, the sequence information of the nucleic acid with the preset sequence is directly identified by the label signal after the nucleic acid with the preset sequence binds to the probe.

In a typical implementation of the present disclosure, the nucleic acid with the preset sequence has M type(s), with the M≥1, and the probe that is reversely complementary to the M type(s) of the nucleic acid with the preset sequence each carries a preset fluorescent label. The sequence information of the nucleic acid with the preset sequence can be identified only by the presence or absence of the fluorescence signal, which can greatly shorten the sequencing time and reduce the sequencing cost.

In some preferred embodiments, the method includes: S1, immobilizing the nucleic acid with the preset sequence on a solid support; S2, dividing the probe into N group(s), with the N≥1, where each group of the probe includes a type(s) of the probe, with the a ≥1, and the a type(s) of the probe in the group each carries a different fluorescent label; and hybridizing a first group of the probe carrying a fluorescent label with the nucleic acid with the preset sequence to collect a fluorescent signal; S3, stripping the first group of the probe, and hybridizing a second group of the probe with the nucleic acid with the preset sequence to collect a fluorescence signal; S4, repeating the step S3 until all the nucleic acid with the preset sequence is detected; and S5, identifying the sequence information of the nucleic acid with the preset sequence according to presence or absence of the hybridization signal between the probe and the nucleic acid.

Typically, the nucleic acid with the preset sequence is an index.

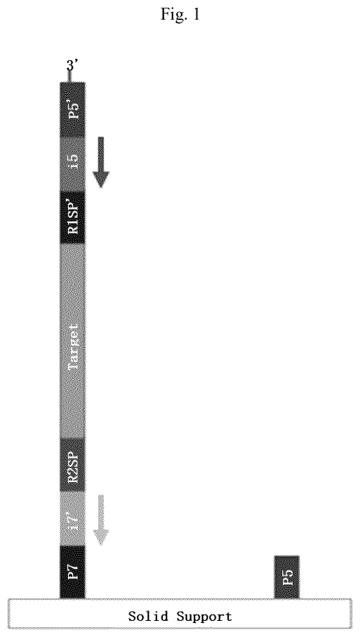

In an embodiment of the present disclosure, the construction of a DNA sequencing library includes: 1) randomly breaking the DNA to be detected into fragments of a certain length; and 2) processing the two ends of the fragments. As shown in FIG. 1, each resulting DNA single strand has P7, i7 index, and Rd2SP (Read2 Sequencing Primer) at the 5′ end, and P5′, i5 index, and Rd1SP (Read1 Sequencing Primer) at the 3′ end. These DNA single strands form a sample library.

The sequences of the above indexes are known, and the index probe (1 or 2) is labeled with 4 fluorescent dyes (AF532, Rox, Cy5, and AF700). Probes that can bind to the indexes of known sequences are synthesized, and each probe carries a preset fluorescent label. When the fluorescent label after the index is bound to the probe emits a fluorescent signal, the sequence information of the index can be identified through the presence or absence of the fluorescent signal, that is, which index is identified.

Optionally, in other embodiments, the number of indexes (or samples) used is entirely identified by the implementer. For example, if the implementer wants to use 40 indexes, 10 rounds of hybridization (40 indexes divided by 4 fluorescence per round) are required to identify the sequence information of different indexes.

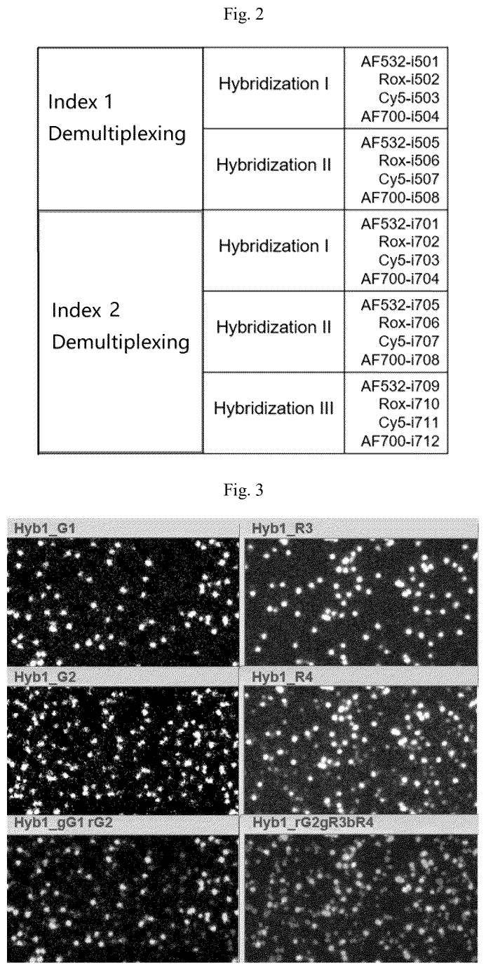

In the embodiment of FIG. 1, conventional i5 and i7 are used as index examples. As shown in FIG. 2, 8 index probes can be used for i5 (i501, i502, . . . , i508), requiring 2 rounds of hybridization (8 divided by 4), and 12 index probes can be used for i7 (i701, i702, . . . , i712), requiring 3 rounds of hybridization (12 divided by 4). The index is usually of 8 to 12 bp, and of course, it can be longer. In the legend, both i5 and i7 are of 8 bp.

It can be understood that the use of AF532, Rox, Cy5, and AF700 for the fluorescent label described above is only illustrative, and other dyes or fluorescent labels can also achieve this purpose.

The method for nucleic acid sequence detection of this disclosure can be applied to the identification of tag sequences in the current nucleic acid sequencing. The sequencing can be single-ended sequencing, double-ended sequencing, etc. of the second-generation sequencing, the sequencing template can include one or more preset sequences, etc., and the sequencing method can be performed by means of bridge amplification or rolling circle amplification.

According to a typical implementation of this disclosure, a sequencing method is provided, and the method includes sequencing of a sequence to be detected with an unknown sequence and sequencing of a preset sequence as an index, where the sequencing of the preset sequence includes identifying sequence information of the preset sequence using the above methods in this disclosure. The preset sequence can be an index sequence, which can be identified using the above method for nucleic acid sequence detection in this disclosure.

In a typical embodiment of this disclosure, a single-stranded template nucleotide for sequencing sequentially includes a first linker sequence region linked to a solid support, a first index region, a first sequencing primer region, a sequence region to be detected, a second sequencing primer region, a second index region, and a second linker sequence region linked to the solid support from a 5′ end to a 3′ end; and the method includes: immobilizing the single-stranded template nucleotide on the solid support through a bridge reaction to form a cluster, removing one of nucleotide strands identical or complementary to the single-stranded template nucleotide by cleaving, and retaining a first sequencing strand; hybridizing a probe carrying a fluorescent label for identifying the first index region with a nucleic acid of the first index region to collect a fluorescent signal and identify a sequence of the first index region, and then stripping the probe; hybridizing a first nucleic acid fragment covering a portion of the first linker sequence region, the first index region and the first sequencing primer region with the cluster, linking the first nucleic acid fragment to a linker sequence on the solid support, and partially sequencing the sequence to be detected with the first nucleic acid fragment as a primer, and then extending the first nucleic acid fragment to the second linker sequence region; removing the first sequencing strand; hybridizing a probe carrying a fluorescent label for identifying the second index region with a nucleic acid of the second index region to collect a fluorescent signal and identify a sequence of the second index region, and then stripping the probe; and performing complementary strand sequencing of the sequence to be detected using a second nucleic acid fragment complementary to the second sequencing primer region as a prime.

Usually, the SBS method takes several hours for index sequencing, while the color development of the fluorescently labeled probe in this disclosure can be completed within a few minutes, so the entire sequencing workflow (i.e., turnover time) can be significantly shortened. Meanwhile, the manufacturing process of fluorescently labeled oligonucleotides is mature, the method is simple, and the price is low.

In the implementation of the sequencing method of this disclosure, other steps can be achieved using some conventional technical means in the art. However, in order to improve the efficiency and accuracy of overall sequencing, preferably, removing one of nucleotide strands identical or complementary to the single-stranded template nucleotide by cleaving includes: cleaving a uracil base or an 8-oxoguanine base on a nucleic acid linker on the solid support to form a nick, and removing a nucleotide strand with the nick using a formamide solution; and typically, the uracil base can be cleaved using a uracil hydrolase, or the 8-oxoguanine base can be cleaved using an Fpg glycosidase, and a phosphate group is generated at a 3′ end. The probe can be stripped with a formamide solution, and then whether the probe is completely stripped is identified by fluorescence observation.

In a typical embodiment of this disclosure, both a 5′ end and a 3′ end of the first nucleic acid fragment are phosphorylated, and the step of linking the first nucleic acid fragment to a linker sequence on the solid support, and sequencing the sequence to be detected with the first nucleic acid fragment as a primer specifically includes: linking the first nucleic acid fragment to the linker sequence on the solid support using a DNA ligase, dephosphorylating the 3′ end of the first nucleic acid fragment using a phosphatase, and sequencing the sequence to be detected with the first nucleic acid fragment as a primer. Similarly, after removing the first sequencing strand, the method further includes a step of blocking a free 3′-hydroxyl group with a terminator or phosphorylation, and optionally, the terminator is a dideoxynucleotide.

The beneficial effects of the present disclosure will be explained in further detail below in conjunction with specific embodiments.

### Embodiment 1

In this embodiment, a sequencer could measure 4 fluorescent dyes at a time.

The probe sequence used in this embodiment was as follows:

AF532-i501:
(SEQ ID NO: 1)
5′-AF532-TCT ACA CTA TAG CCT-3′;
Rox-i502:
(SEQ ID NO: 2)
5′-Rox-TCT ACA CAT AGA GGC-3′;
Cy5-i503:
(SEQ ID NO: 3)
5′-Cy5-TCT ACA CCC TAT CCT-3′;
Atto700-i504:
(SEQ ID NO: 4)
5′-Atto700-TCT ACA CGG CTC TGA-3′;
AF532-i505:
(SEQ ID NO: 5)
5′-AF532-TCT ACA CAG GCG AAG-3′;
Rox-i506:
(SEQ ID NO: 6)
5′-Rox-TCT ACA CTA ATC TTA-3′;
Cy5-i507:
(SEQ ID NO: 7)
5′-Cy5-TCT ACA CCA GGA CGT-3′;
and
Atto700-i508:
(SEQ ID NO: 8)
5′-Atto700-TCT ACA CGT ACT GAC-3′.

The construction of a DNA sequencing library included: 1) randomly breaking the DNA to be detected into fragments of a certain length; and 2) processing the two ends of the fragments. As shown in FIG. 1, each resulting DNA single strand had P7, i7 index′, Rd2SP (Read2 Sequencing Primer), the fragment to be detected Rd1SP′ (Read1 Sequencing Primer), i5 index, and P5′ from the 5′ end to 3′ end, respectively. These DNA single strands formed a sample library.

The sequences of the above indexes were pre-set and known. Probes that could bind to the indexes of known sequences were synthesized, and each probe carried a preset fluorescent label. When the fluorescent label after the index was bound to the probe emitted a fluorescent signal, the sequence information of the index could be identified through the presence or absence of the fluorescent signal, i.e., which index it was could be identified.

Optionally, in other embodiments, the number of indexes (or samples) used is entirely identified by the implementer. For example, if the implementer wants to use 40 indexes and 4 fluorescent labels, then 10 sets of index probes (40 divided by 4) are required for 10 rounds of hybridization in sequencing to identify the sequence information of different indexes.

In the embodiment in FIG. 1, conventional i5 and i7 were used as index examples. As shown in FIG. 2, 8 indexes could be used for i5 (i501, i502, . . . , i508), requiring 2 rounds of hybridization (8 divided by 4), and 12 indexes could be used for i7 (i701, i702, . . . , i712), requiring 3 rounds of hybridization (12 divided by 4). The index was usually of 8 to 12 bp, and of course, it could be longer. For example, both i5 and i7 were of 8 bp.

In this way, the sequence information of the index can be identified only by the presence or absence of the fluorescence signal, which can greatly shorten the sequencing time and reduce the sequencing cost. Usually, the SBS method takes several hours for index sequencing, while the color development of the fluorescently labeled probe can be completed within a few minutes, so the entire sequencing workflow (i.e., turnover time) can be significantly shortened. Meanwhile, the manufacturing process of fluorescently labeled oligonucleotides is mature, the method is simple, and the price is low. In other words, using the technical solutions of this disclosure, there is no longer a need to sequence the index, and the sequence information of the index can be confirmed only through inexpensive probe hybridization and fluorescence reactions.

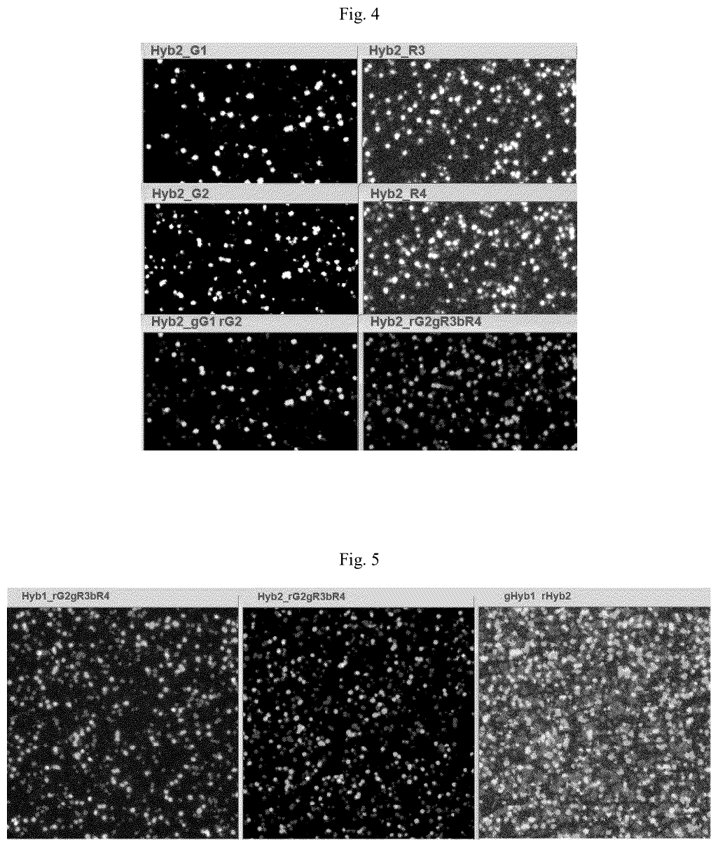

In FIG. 3, demultiplexing (sequence confirmation) of i5 and i7 by the index1 probe and the index2 probe, respectively, is shown. Hybridization reactions of probes labeled with fluorescent groups AF532-i501, Rox-i502, Cy5-i503, and Af700-i504 would identify the sequences of indexes i501, i502, i503, and i504, respectively. FIG. 3 lists four channel images (Hybl_G1, Hybl_G2, Hybl_R3, Hybl_R4), showing that the indexes i501, i502, i503, and i504 were identified. In the composite image of Hybl_gG1rG2, yellow represented the index i501, and red represented the index i502. In the composite image of Hybl_rG2gR3bR4, red represented the index i501 or i502 (note: identified in Hybl_gG1rG2), light blue represented the index i503, and blue represented the index i504.

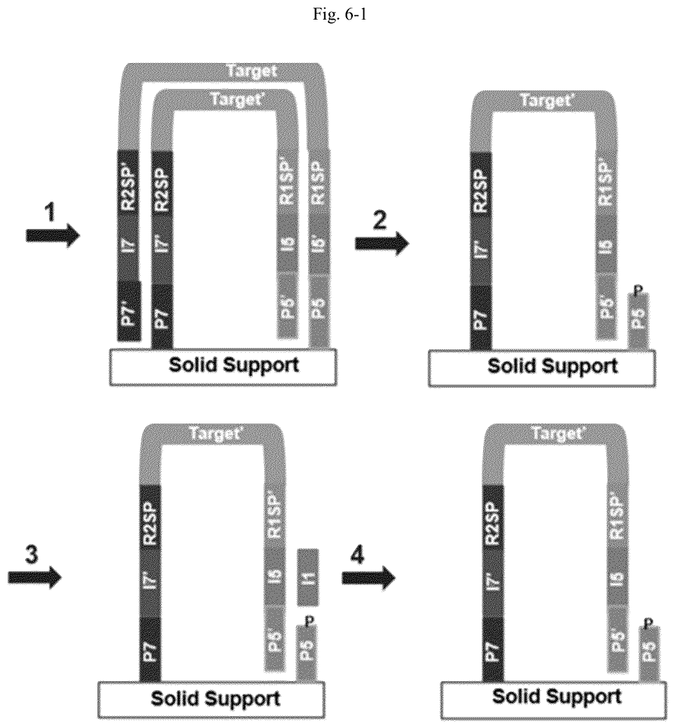

After dehybridization, a second cycle of hybridization with probes AF532-i505, Rox-506, Cy5-i507 and AF700-i508 would identify indexes i505, i506, i507, and i508, respectively. FIG. 4 shows four channel images (Hyb2_G1, Hyb2_G2, Hyb2_R3, Hyb2_R4), showing that the indexes i501, i502, i503 and i504 were identified. In the composite image of Hyb2_gG1rG2, yellow represented the index i505, and red represented the index i506. In the composite image of Hyb2_rG2gR3bR4, red represented the index i505 or i506 (note: must be identified in Hyb2_gG1rG2), light blue represented the index i507, and blue represented the index i508.

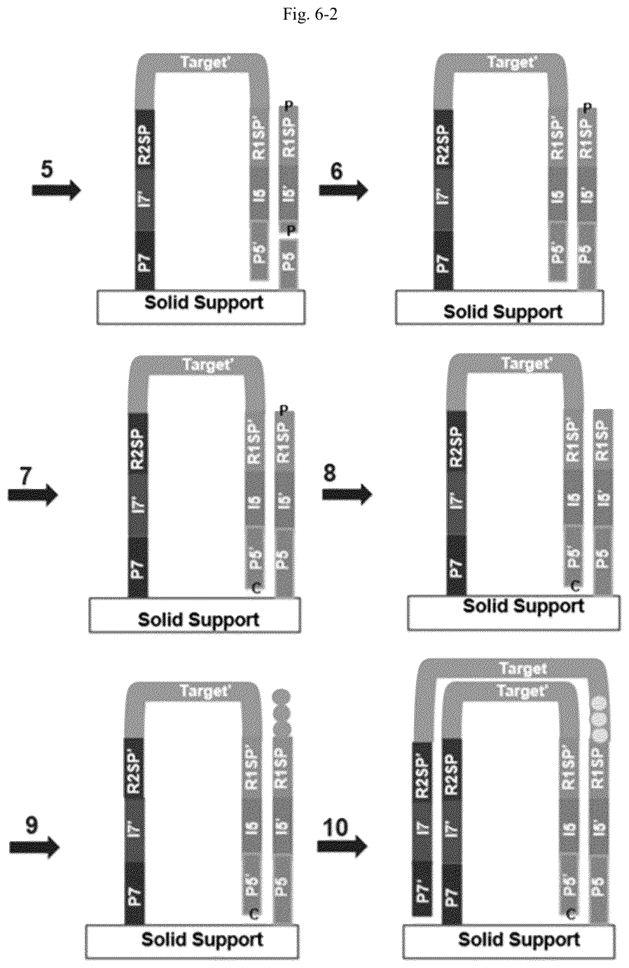

In FIG. 5, all readings from the first round of hybridization were labeled in green (indexes i501, i502, i503, and i504), while all readings from the second round of hybridization were labeled in red (indexes i505, i506, i507, and i508). From the composite image on the far right of FIG. 5, it was evident that almost all readings were correctly recognized, while only a small portion of yellow could not be recognized, which was a composite color of green and red. These readings may be a mixture between different clusters, possibly due to excessive clustering in this experiment.

Identification of the index 2 could similarly be achieved by three rounds of hybridization. Four or more dyes could be used. For example, i5 could be demultiplexed with 8 or fewer dyes, but each index was encoded with two or more dyes. In this case, only one hybridization was performed to identify index 1 or index 2.

### Embodiment 2

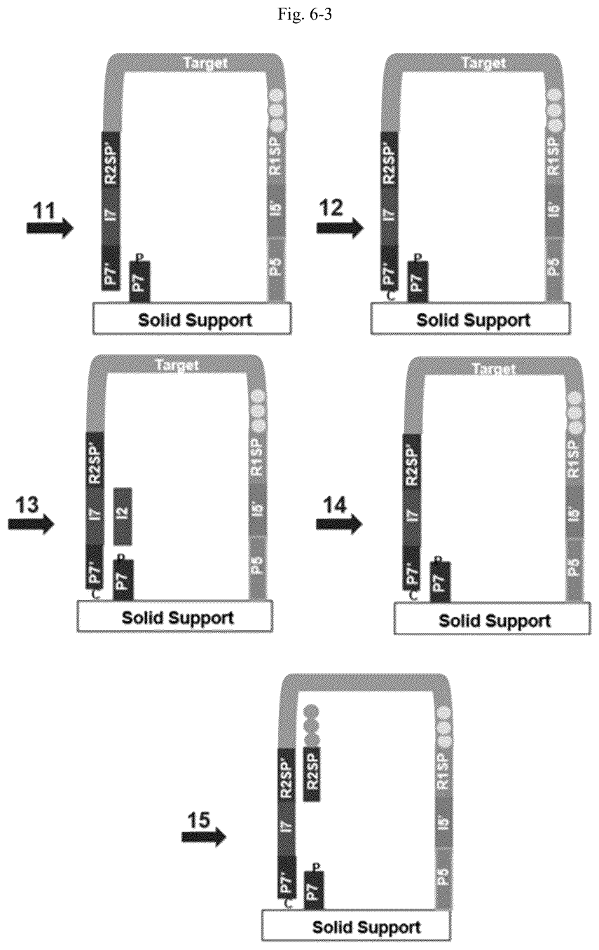

This embodiment illustrates the disclosure of the method for nucleic acid sequence detection of this disclosure in sequencing, and provides a novel sequencing method. Please refer to FIGS. 6-1, 6-2, and 6-3 for the steps.

Step 1, through the typical bridge PCR, clusters were formed.

Step 2, cleavage of the Uracil base on P5 to create a nick on the P5 strand. Condition: 40 U/mL Enzyme at 37° C. (6 min twice) and at 41° C. (6 min twice) (note: total incubation of 24 min). The nicked strand was removed from the flow cell with formamide solution. By the way, we used a uracil hydrolysis enzyme, which generated the fragment with phosphate group at the 3′ end (labeled as P in FIGS. 6-1, 6-2, and 6-3).

Step 3, Index 5 sequencing was demultiplexed with the hybridization probes (I1) (note: using the method in Embodiment 1).

Step 4, the hybridization probes were stripped with formamide solution. The completion of stripping was confirmed by the fact that there were no fluorescent clusters observed anymore during the imaging.

Step 5, the phosphate group at 3′ was removed with a phosphatase, rSAP, from NEB. Condition: 16 U/mL rSAP at 55° C. for 30 min. The oligonucleotide, which covered a small portion of P5, Index 1, and R1SP was hybridized to the clusters. Both 3′- and 5′-ends of the oligonucleotide were phosphorylated.

Step 6, the oligonucleotide was ligated to the 3′ end of the P5 on the solid support. Condition: 20 U/uL Hi-T4 DNA Ligase (NEB) with 2 mM ATP at 25° C. for an hour.

Step 7, all available 3′-hydroxyl group was blocked with terminators, which had no free 3′-hydroxyl group, and for example, could be a deoxyribonucleotide.

Step 8, the phosphate group on the covalent R1SP was cleaved with the same condition as the one at step 5.

Step 9, sequencing with a reversible terminator (a length range of sequencing was 35 to 150 bp). In addition, the 5′ end of the sequencing strand was covalently bound to the solid support, as confirmed by stripping with the formamide solution by the inventor (i.e., unable to remove cluster fluorescence). Because if the sequencing strand was not covalently bound, the fluorescence of the cluster was eliminated during sequencing when incubated at 55-70° C.

Step 10, after removal of the protecting group at the 3′-end, the sequencing strand was fully extended with the typical enzyme. Condition: 320 U/mL Enzyme and 120 uM dNTPs at 65° C. for an hour.

Step 11, there was an oxG on the P7 sequence, so its hydrolysis with Fpg created a nick at the strand. Condition: 230 U/mL Fpg (NEB) at 37° C. for 30 min. Stripping with formamide solution made the clusters only having the opposite strand.

Step 12, all the free 3′-hydroxyl groups were capped with the similar condition in Step 7.

Step 13, Index 7 was demultiplexed through hybridization probes (I2), similarly in Step 3.

Step 14, hybridization probes were stripped with formamide solution.

Step 15, Read 2 sequencing was typically performed. In this case, 35 cycles were run even though much more cycles were possible (a sequencing length range was 35-150 bp).

The foregoing is only a preferred embodiment of the present disclosure and is not intended to limit the present disclosure. For those skilled in the art, the present disclosure may have various modifications and variations. Any modifications, equivalent substitutions, improvements, etc. made within the spirit and principles of the present disclosure shall be included within the scope of protection of the present disclosure.

1. A method for nucleic acid sequence detection, comprising:
providing a nucleic acid with a preset sequence; and
detecting sequence information of the nucleic acid through a hybridization signal between a probe and the nucleic acid.

2. The method according to claim 1, wherein the nucleic acid with the preset sequence has M type(s), with the M≥1, and the probe that is reversely complementary to the M type(s) of the nucleic acid with the preset sequence each carries a preset fluorescent label.

3. The method according to claim 1, comprising:
S1, immobilizing the nucleic acid with the preset sequence on a solid support;
S2, dividing the probe into N group(s), with the N≥1, wherein each group of the probe comprises a type(s) of the probe, with the a ≥1, and the a type(s) of the probe in the group each carries different fluorescent labels; and hybridizing a first group of the probe carrying a fluorescent label with the nucleic acid with the preset sequence to collect a fluorescent signal;
S3, stripping the first group of the probe, and hybridizing a second group of the probe with the nucleic acid with the preset sequence to collect a fluorescence signal;
S4, repeating step S3 until all the nucleic acid with the preset sequence is detected; and
S5, identifying the sequence information of the nucleic acid with the preset sequence according to the presence or absence of the hybridization signal between the probe and the nucleic acid.

4. The method according to claim 1, wherein the nucleic acid with the preset sequence is an index; and
preferably, a length of the nucleic acid with the preset sequence is 8-12 bp.

5. The method according to claim 4, wherein the fluorescent label comprises one or more selected from the group consisting of AF532, Rox, Cy5, or AF700.

6. A sequencing method, comprising sequencing of a sequence to be detected with an unknown sequence and sequencing of a preset sequence as an index, wherein the sequencing of the preset sequence comprises identifying sequence information of the preset sequence using the method according to claim 1.

7. The method according to claim 6, wherein the sequencing method is double-ended sequencing or single-ended sequencing;
and/or a template strand for sequencing comprises one or more preset sequences;
and/or the sequencing method uses bridge amplification or rolling circle amplification for sequencing.

8. The method according to claim 6, wherein a single-stranded template nucleotide for sequencing sequentially comprises a first linker sequence region linked to a solid support, a first index region, a first sequencing primer region, a sequence region to be detected, a second sequencing primer region, a second index region, and a second linker sequence region linked to the solid support from a 5′ end to a 3′ end; and the method comprises:
immobilizing the single-stranded template nucleotide on the solid support through a bridge reaction to form a cluster, removing one of nucleotide strands identical or complementary to the single-stranded template nucleotide by cleaving, and retaining a first sequencing strand;
hybridizing a probe carrying a fluorescent label for identifying the first index region with a nucleic acid of the first index region to collect a fluorescent signal and identify a sequence of the first index region, and then stripping the probe;
hybridizing a first nucleic acid fragment covering a portion of the first linker sequence region, the first index region, and the first sequencing primer region with the cluster, linking the first nucleic acid fragment to a linker sequence on the solid support, and partially sequencing the sequence to be detected with the first nucleic acid fragment as a primer, and then extending the first nucleic acid fragment to the second linker sequence region;
removing the first sequencing strand;
hybridizing a probe carrying a fluorescent label for identifying the second index region with a nucleic acid of the second index region to collect a fluorescent signal and identify a sequence of the second index region, and then stripping the probe; and
performing complementary strand sequencing of the sequence to be detected using a second nucleic acid fragment complementary to the second sequencing primer region as a prime.

9. The method according to claim 8, wherein the removing one of nucleotide strands identical or complementary to the single-stranded template nucleotide by cleaving comprises: cleaving a uracil base or an 8-oxoguanine base on a nucleic acid linker on the solid support to form a nick, and removing a nucleotide strand with the nick using a formamide solution.

10. The method according to claim 8, wherein the uracil base is cleaved using a uracil hydrolase, or the 8-oxoguanine base is cleaved using an Fpg glycosidase.

11. The method according to claim 8, wherein stripping the probe comprises stripping the probe with a formamide solution.

12. The method according to claim 11, wherein the method further comprises determining whether the probe is completely stripped by fluorescence observation.

13. The method according to claim 7, wherein both a 5′ end and a 3′ end of the first nucleic acid fragment are phosphorylated.

14. The method according to claim 13, wherein the step of linking the first nucleic acid fragment to a linker sequence on the solid support, and sequencing the sequence to be detected with the first nucleic acid fragment as a primer specifically comprises:
linking the first nucleic acid fragment to the linker sequence on the solid support using a DNA ligase, dephosphorylating the 3′ end of the first nucleic acid fragment using a phosphatase, and sequencing the sequence to be detected with the first nucleic acid fragment as a primer.

15. The method according to claim 6, wherein after removing the first sequencing strand, the method further comprises a step of blocking a free 3′-hydroxyl group with a terminator or phosphorylation, and optionally, the terminator is a dideoxynucleotide.


SEQUENCE LISTING (8 sequences)

SEQ ID NO: 1 - DNA, 15 bp, synthetic construct
  source 1..15: mol_type = other DNA
  modified_base 1: mod_base = OTHER; note = Alexa Fluor 532
tctacactatagcct

SEQ ID NO: 2 - DNA, 15 bp, synthetic construct
  source 1..15: mol_type = other DNA
  modified_base 1: mod_base = OTHER; note = Rhodamine X
tctacacatagaggc

SEQ ID NO: 3 - DNA, 15 bp, synthetic construct
  source 1..15: mol_type = other DNA
  modified_base 1: mod_base = OTHER; note = Sulfo-Cyanine5
tctacaccctatcct

SEQ ID NO: 4 - DNA, 15 bp, synthetic construct
  source 1..15: mol_type = other DNA
  modified_base 1: mod_base = OTHER; note = ATTO-700
tctacacggctctga

SEQ ID NO: 5 - DNA, 15 bp, synthetic construct
  source 1..15: mol_type = other DNA
  modified_base 1: mod_base = OTHER; note = Alexa Fluor 532
tctacacaggcgaag

SEQ ID NO: 6 - DNA, 15 bp, synthetic construct
  source 1..15: mol_type = other DNA
  modified_base 1: mod_base = OTHER; note = Rhodamine X
tctacactaatctta

SEQ ID NO: 7 - DNA, 15 bp, synthetic construct
  source 1..15: mol_type = other DNA
  modified_base 1: mod_base = OTHER; note = Sulfo-Cyanine5
tctacaccaggacgt

SEQ ID NO: 8 - DNA, 15 bp, synthetic construct
  source 1..15: mol_type = other DNA
  modified_base 1: mod_base = OTHER; note = ATTO-700
tctacacgtactgac